(19) United States
(12) Patent Application Publication
(10) Pub. No.: US 2021/0102226 A1
(43) Pub. Date: Apr. 8, 2021

(54) Emulsifier Composition Obtainable from Free Fatty Acids

(71) Applicant: Bunge Loders Croklaan B.V., Wormerveer (NL)

(72) Inventor: Jun Ma, Wormerveer (NL)

(73) Assignee: Bunge Loders Croklaan B.V., Wormerveer (NL)

(21) Appl. No.: 16/499,583

(22) PCT Filed: May 4, 2018
(86) PCT No.: PCT/EP2018/061616

§ 371 Date: Sep. 30, 2019

(30) Foreign Application Priority Data
May 8, 2017 (EP) 17275062.2

(51) Int. Cl. C12P 7/64; B01F 17/00; A23L 29/10; A23D 7/00; A23D 7/01; A23D 7/04; A21D 2/16; A21D 13/80; A23G 3/40

(52) U.S. Cl. CPC C12P 7/6454; B01F 17/0021; B01F 17/0092; A23L 29/10; A23D 7/003; A23V 2002/00; A23D 7/04; A21D 2/165; A21D 13/80; A23G 3/40; A23D 7/011

(57) ABSTRACT

A method for preparing a non-hydrogenated, non-palm emulsifier composition, comprises the steps of:—providing a fatty acid composition comprising at least 80% by weight free fatty acids, and—reacting the fatty acid composition with glycerol, wherein the emulsifier composition comprises: at least 20% by weight monoglycerides; less than 60% by weight of diglycerides; and and from 0-80% by weight triglycerides, wherein the weight % is with respect to the total of monoglycerides, diglycerides and triglycerides, and wherein the fatty acid residues bound to the monoglycerides, diglycerides and triglycerides in the emulsifier composition comprise: from 5% to 80% by weight stearic acid (C18:0); from 10% to 80% by weight oleic acid (C18:1); and from 1% to 10% by weight palmitic acid (C16:0), based on the total weight of C8 to C24 fatty acids.

This invention relates to emulsifier compositions, to the use of the compositions as an emulsifier and to methods for preparing the composition.

Emulsifiers are used in many applications where it is desired to mix two generally immiscible phases, which are typically aqueous and non-aqueous. Emulsifiers find use in the food industry as well as in other applications such as cosmetics.

Compounds having a relatively polar moiety and a non-polar moiety may have emulsifying properties. Lecithin is an emulsifier that is commonly used in the food industry. Mono- and di-glycerides are also examples of emulsifiers. For example, monoglycerides are used as emulsifiers in the confectionery fillings described in EP-A-0547658.

WO 2014/020114 relates to a fat blend composition for a fat spread for lowering cholesterol levels. The fatty acid composition of the blend comprises 20% or less saturated fatty acids, of which 38% or less are palmitic acid and 20% or more are stearic acid.

WO 2015/150405 discloses a free fatty acid composition which comprises'. greater than 60% by weight stearic acid; from 3 to 30% by weight oleic acid; and less than 10% by weight palmitic acid. The composition may be used in the preparation of a triglyceride.

US 2016/0008262 describes a composition comprising the product from a reaction of a natural butter or natural oil such as shea butter with glycerin in the presence of a basic catalyst. The reaction products retain the unsaponifiable portion of the natural butter or natural oil. The products are self-emulsifiable and are described as being useful in personal care, cosmetic, pharmaceutical, paper and textile applications.

There is a need for improved emulsifiers. In particular, there is a need for emulsifiers that can be readily produced from convenient sources and/or that can increase the stability of an emulsion against separation into its constituent phases.

According to the present invention, there is provided a method for preparing a non-hydrogenated, non-palm emulsifier composition, comprising the steps of:
providing a fatty acid composition comprising at least 80% by weight free fatty acids, and
reacting the fatty acid composition with glycerol,

wherein the emulsifier composition comprises:
at least 20% by weight monoglycerides;
less than 60% by weight of diglycerides; and
and from 0-80% by weight triglycerides,
wherein the weight % is with respect to the total of monoglycerides, diglycerides and triglycerides, and

wherein the fatty acid residues bound to the monoglycerides, diglycerides and triglycerides in the emulsifier composition comprise:
from 5% to 80% by weight stearic acid (C18:0);
from 10% to 80% by weight oleic acid (C18:1); and
from 1% to 10% by weight palmitic acid (C16:0),
based on the total weight of C8 to C24 fatty acids.

In another aspect relating to a first composition, the invention provides a non-hydrogenated, non-palm emulsifier composition comprising:
at least 20% by weight monoglycerides;
less than 60% by weight of diglycerides; and
from 0-80% by weight triglycerides,
wherein the weight % is with respect to the total of monoglycerides, diglycerides and triglycerides, and

wherein the fatty acid residues bound to the monoglycerides, diglycerides and triglycerides in the emulsifier composition comprise:
from 5% to 40% by weight stearic acid (C18:0);
from 45% to 80% by weight oleic acid (C18:1); and
from 1% to 10% by weight palmitic acid (C16:0),
based on the total weight of C8 to C24 fatty acids.

In yet another aspect relating to a second composition, the invention provides a non-hydrogenated, non-palm emulsifier composition comprising:
at least 20% by weight monoglycerides;
less than 60% by weight of diglycerides; and
and from 0-80% by weight triglycerides,
wherein the weight % is with respect to the total of monoglycerides, diglycerides and triglycerides, and

wherein the fatty acid residues bound to the monoglycerides, diglycerides and triglycerides in the emulsifier composition comprise:
from 50% to 90% by weight stearic acid (C18:0);
from 10% to 30% by weight oleic acid (C18:1); and
from 1% to 10% by weight palmitic acid (C16:0),
based on the total weight of C8 to C24 fatty acids.

In another aspect., the invention provides the use of an emulsifier composition of the invention as an emulsifier in a food application, such as bakery or confectionery, preferably in puff pastry, cake, Danish rolls or water based fillings.

It has surprisingly been found that emulsifier compositions prepared according to the invention can provide emulsions having improved stability compared, for example, to conventional emulsifier compositions.

The term “fatty acid”, as used herein, refers to straight chain saturated or unsaturated (including mono- and poly-unsaturated) carboxylic acids having from 8 to 24 carbon atoms (C8 to C24). A fatty acid having n carbon atoms and x double bonds may be denoted Cn:x. For example, palmitic acid may be denoted C16:0 and oleic acid may be denoted C18:1. Percentages of fatty acids in compositions referred to herein include acyl groups in tri-, di- and mono-glycerides present in the glycerides as is customary terminology in the art and are based on the total weight of C8 to C24 fatty acids. The fatty acid profile (i.e., composition) may be determined, for example, by fatty acid methyl ester analysis (FAME) using gas chromatography according to ISO 12966-2 and ISO 12966-4.

The compositions of the invention are non-hydrogenated. This means that the compositions are not prepared or derived from a fat that has been subjected to hydrogenation to convert unsaturated fatty acyl groups to saturated fatty acyl groups. The requirement for the fat to be non-hydrogenated means that the content of trans fatty acid residues in the composition is typically less than 1% by weight based on total C8 to C24 fatty acids present, more preferably not more than 0.5% by weight.

The term “fat” refers to glyceride fats and oils containing fatty acid acyl groups and does not imply any particular melting point. The term “oil” is used synonymously with “fat”. Fats predominantly comprise triglycerides.

The method of the invention uses as one of the starting materials a fatty acid composition comprising at least 80% by weight free fatty acids, preferably at least 90% by weight free fatty acids. The fatty acid composition comprises at least stearic, oleic, linoleic and palmitic acids and typically will also contain other fatty acids. Fatty acids other than stearic, oleic, linoleic and palmitic are typically present in the composition at a level of less than 10% by weight, more preferably less than 8% by weight, such as less than 5% by weight, based on total C8 to C24 fatty acids present.

In the method of the invention, the fatty acid composition reacts with glycerol to form glycerides i.e., monoglycerides, diglycerides and triglycerides.

Preferably, the fatty acid composition is from at least one non-palm source selected from shea, sal, mango or combinations thereof. Thus, the emulsifier compositions are preferably obtainable from shea, sal, mango and combinations thereof. The non-palm source may be a butter (as obtained naturally) or a fraction thereof, such as a stearin or olein fraction. Most preferred non-palm sources are shea olein, shea stearin and sal olein, with shea olein being particularly preferred.

The term non-palm refers to fat or oil products that are not obtained from oil palm species, including, for example, the African oil palm Elaeis guineensis, the American oil palm Elaeis oleifera and the maripa palm Attalea maripa.

Preferably, the fatty acid composition is obtained from shea butter, shea olein, shea stearin, sal butter, sal stearin, sal olein, mango butter, mango stearin, mango olein or mixtures thereof, preferably shea olein. For example, the fatty acid composition may be a fatty acid distillate, such as from shea olein. Shea olein typically contains about 10-15% free fatty acids. Distillation may be carried out, for example, at a temperature of from 150 to 250° C. under a reduced pressure such as of 0.001 to 1 mbar. The distillate may be further treated to alter the fatty acid composition, for example by fractionation e.g., dry fractionation. The fraction that is used in the method of the invention may be an olein fraction or a stearin fraction, preferably an olein fraction that is enriched in oleic acid relative to the starting distillate.

A preferred fatty acid composition is obtained by distillation of shea olein and comprises, based on the total weight of C8 to C24 fatty acids:

1-10% palmitic acid;

25-50% stearic acid;

40-60% oleic acid;

5-15% linoleic acid; and

less than 10% of other fatty acids.

Another preferred fatty acid composition is obtained by distillation of shea olein, followed by fractionation to form an olein fraction (obtained, for example, as the liquid fraction by cooling a molten mixture to a temperature in the range of from 25 to 50° C.) and comprises, based on the total weight of C8 to C24 fatty acids:

1-10% palmitic acid;

5-25% stearic acid;

60-80% oleic acid;

5-15% linoleic acid; and

less than 10% of other fatty acids.

A further preferred fatty acid composition is obtained by distillation of shea olein, followed by fractionation to form a stearin fraction (obtained, for example, as the solid fraction by cooling a molten mixture to a temperature in the range of from 25 to 50° C.) and comprises, based on the total weight of C8 to C24 fatty acids:

1-10% palmitic acid;

60-80% stearic acid;

10-30% oleic acid;

1-10% linoleic acid; and

less than 10% of other fatty acids.

The reaction of the fatty acid composition with glycerol in the method of the invention may be carried out in the presence of an enzymatic catalyst, such as a lipase, preferably from Candida Antarctica, most preferably Candida antarctica lipase B. The lipase may be immobilised. Candida antarctica lipase B immobilized on acrylic resin is available as Novozym 435.

The conditions for reacting the fatty acid composition with glycerol in the presence of the enzyme preferably comprise a weight ratio of fatty acid composition to glycerol in the range of from 10:1 to 2:1, more preferably from 5:1 to 3:1. The reaction is carried out typically for 10 to 48 hours at a suitable temperature for the enzyme, preferably in the range of 55° C. to 70° C.

Alternatively, the reaction of the fatty acid composition with glycerol may be carried out in the presence of an inorganic salt. The inorganic salt is typically not a salt of a strong base such as a metal hydroxide, oxide or alkoxide. Preferably the salt is a divalent metal halide, more preferably zinc chloride.

Conditions for reacting the fatty acid composition with glycerol in the presence of the salt preferably comprise a weight ratio of fatty acid composition to glycerol in the range of from 10:1 to 1:2, more preferably from 3:1 to 1:1. The reaction is typically carried out for 10 minutes to 2 hours at a temperature in the range of 150° C. to 250° C. at a reduced pressure of from 5 to 500 mbar.

The method of the invention, when catalyzed either by an enzyme or an inorganic salt, may comprise one or more further refining steps. For example, the method may comprise the further steps of bleaching and deodorization after the reaction of the fatty acid with glycerol. Preferably, deodorization is carried out at a reduced pressure in the range of 0.5 mbar to 2 mbar at a temperature in the range of from 130° C. to 170° C.

The method of the invention may also comprise a further step of distillation of the product. The distillate thus formed will have an increased level of monoglycerides relative to diglycerides and triglycerides compared to the product before distillation.

First Composition

In one embodiment, the invention provides a first composition which is a non-hydrogenated, non-palm emulsifier composition comprising:
at least 20% by weight monoglycerides;
less than 60% by weight of diglycerides; and
from 0-80% by weight triglycerides,
wherein the weight % is with respect to the total of monoglycerides, diglycerides and triglycerides, and

wherein the fatty acid residues bound to the monoglycerides, diglycerides and triglycerides in the emulsifier composition comprise:
from 5% to 40% by weight stearic acid (C18:0);
from 45% to 80% by weight oleic acid (C18:1); and
from 1% to 10% by weight palmitic acid (C16:0),
based on the total weight of C8 to C24 fatty acids.

The first composition is typically produced from fatty acid compositions obtained directly from the distillation of shea olein, optionally followed by fractionation to form an olein fraction.

Preferably, the weight ratio of stearic acid to oleic acid in the first composition is from 1:8 to 9:10. It will be appreciated that the fatty acid residues of the emulsifier composition of the invention refers to acyl groups that are present as acyl groups bonded in monoglycerides, diglycerides and/or triglycerides.

In one embodiment, the first composition preferably comprises from 20% to 50% by weight monoglycerides, preferably from 22% to 40% by weight monoglycerides. Additionally, or alternatively, the composition may comprise from 30% to 60% by weight diglycerides. The triglyceride content is preferably less than 40% by weight, more preferably less than 30% by weight. All percentages in this paragraph being with respect to the total of monoglycerides, diglycerides and triglycerides.

In another embodiment, the first composition comprises: at least 85% by weight monoglycerides; less than 10% by weight of diglycerides; and less than 5% by weight triglycerides with respect to the total of monoglycerides, diglycerides and triglycerides. Typically, this composition is obtained by a further step of distillation of the product of the method of the invention and collection of the distillate.

Preferably, the first composition comprises in the glycerides: from 1% to 12% by weight linoleic acid (C18:2), preferably from 1 to 10% by weight linoleic acid.; and/or

from 10% to 35% by weight stearic acid; and/or

from 50% to 70% by weight oleic acid; and/or

from 4.5% to 8% by weight palmitic acid,

based on the total weight of C8 to C24 fatty acids.

Second Composition

The invention also provides a second composition, which is a non-hydrogenated, non-palm emulsifier composition comprising:
at least 20% by weight monoglycerides;
less than 60% by weight of diglycerides; and
and from 0-80% by weight triglycerides,
wherein the weight % is with respect to the total of monoglycerides, diglycerides and triglycerides, and

wherein the fatty acid residues bound to the monoglycerides, diglycerides and triglycerides in the emulsifier composition comprise:

from 50% to 90% by weight stearic acid (C18:0);

from 10% to 30% by weight oleic acid (C18:1); and

from 1% to 10% by weight palmitic acid (C16:0),

based on the total weight of C8 to C24 fatty acids.

The second composition is typically produced from fatty acid compositions obtained from the distillation of shea olein, followed by fractionation to form a stearin fraction. It differs from the first composition by, amongst other things, a higher stearic acid content.

Preferably, the weight ratio of stearic acid to oleic acid in the second composition is from 2:1 to 5:1. It will be appreciated that the fatty acid residues of the emulsifier composition of the invention refers to acyl groups that are present as acyl groups bonded in monoglycerides, diglycerides and/or triglycerides.

The second composition preferably comprises from 15% to 50% by weight monoglycerides, preferably from 20% to 40% by weight monoglycerides. Additionally, or alternatively, the composition may comprise from 40% to 60% by weight diglycerides. The triglyceride content is preferably less than 40% by weight, more preferably less than 30% by weight. All percentages in this paragraph being with respect to the total of monoglycerides, diglycerides and triglycerides.

Preferably, the second composition comprises in the glycerides: from 1% to 8% by weight linoleic acid (C18:2), preferably from 1 to 5% by weight linoleic acid.; and/or from 60% to 80% by weight stearic acid; and/or from 10% to 30% by weight oleic acid; and/or from 1% to 8% by weight palmitic acid, based on the total weight of C8 to C24 fatty acids.

Thus, a particularly preferred second composition comprises: from 15 to 50% by weight monoglycerides;

from 40 to 60% by weight of diglycerides; and less than 30% by weight triglycerides,

wherein the weight % is with respect to the total of monoglycerides, diglycerides and triglycerides, and

wherein the fatty acid residues bound to the monoglycerides, diglycerides and triglycerides in the emulsifier composition comprise:

from 1% to 8% by weight linoleic acid (C18:2), preferably from 1 to 5% by weight linoleic acid.; and/or

from 60% to 80% by weight stearic acid; and/or

from 10% to 30% by weight oleic acid; and/or

from 1% to 8% by weight palmitic acid,

based on the total weight of C8 to C24 fatty acids.

Uses of the Compositions

The invention also provides the use of an emulsifier composition of the invention as an emulsifier in a food application, such as bakery or confectionery. The use of the emulsifier may be for increasing the stability of an emulsion, such as a water-in-oil emulsion.

Emulsifier compositions of the invention may therefore be used in food applications. Examples of food applications are bakery or confectionery, preferably puff pastry, cake, Danish rolls or water based fillings.

Emulsifier compositions of the invention may, for example, be used in the production of bakery products. The bakery products may have a laminated structure. For bakery applications, the emulsifier composition is typically used in conjunction with a fat. Margarine, bakery fat or puff pastry (i.e., laminating) fat and an emulsifier composition of the invention may be combined with flour and water to form a dough. The dough preferably comprises flour in an amount of from 30 to 60% by weight, water in an amount of from 10 to 40% by weight, the margarine, bakery fat or puff pastry (i.e., laminating) fat in an amount of from 10 to 50% by weight and from 0.04% to 0.75% by weight of an emulsifier composition of the invention based on the weight of the dough. Optionally, one or more further ingredients such as salt and flour modifier may be included in the dough. Bakery products are made from dough. The dough preferably has a laminated structure. The bakery products include, for example, puff pastry, croissants, Danish pastries and pies.

Doughs comprising the emulsifier compositions, and a margarine, laminating fat and/or a bakery fat, may be refrigerated, frozen or otherwise stored prior to use. The frozen dough may be packaged and sold to the consumer. In order to form a bakery product, the dough is baked, preferably in an oven. Suitable times and temperatures for baking specific bakery products will be well-known to those skilled in the art.

Cake may be made from a batter that is baked. Cake batters typically comprise, in addition to the emulsifier composition, fat, sugar, flour, milk and eggs. The amount of emulsifier in the batter is typically in the range of 0.6% to 5.6% by weight.

A confectionery filling that is water based may comprise a fat-continuous emulsion with a fat content of 5-50 wt.% while the water content of the remainder is 10-60 wt.%, and the remainder further consists of 90-40 wt.% of at least one of the following components: acidity regulator, thickener, bulking agent, sweetener, flavour, colourant, humectant and preservative, together with the emulsifier. The emulsifier composition of the invention is typically present in an amount of from 0.08% to 1% by weight. The filling will preferably be used in encapsulated form when applied in confectionery products. By the expression “encapsulated” is meant that the filling is surrounded by a coating layer or shell, preferably consisting of, or containing, chocolate. In these cases, the shell, in particular the chocolate shell, represents 20-50 wt % of the total product.

The listing or discussion of an apparently prior-published document in this specification should not necessarily be taken as an acknowledgement that the document is part of the state of the art or is common general knowledge.

Preferences and options for a given aspect, embodiment, feature or parameter of the invention should, unless the context indicates otherwise, be regarded as having been disclosed in combination with any and all preferences and options for all other aspects, embodiments, features and parameters of the invention. In particular, it will be appreciated that features of the method of the invention apply to the emulsifier compositions of the invention and vice versa.

The following non-limiting examples illustrate the invention and do not limit its scope in any way. In the examples and throughout this specification, all percentages, parts and ratios are by weight unless indicated otherwise.

### EXAMPLES

### Example 1

Crude shea olein is obtained after solvent fractionation of crude shea butter. Crude shea olein naturally contains approximatively 10-15% free fatty acids. The free fatty acids were distilled by means of short path distillation at a temperature of about 190° C. to 205° C. and a pressure of about 1×10⁻³ to 8×10⁻³mbar. The free fatty acids were collected as distillate. The fatty acid composition of the products is given in the following Table 1.

| | |
|---|---|
| C8:0 | 0 |
| C10:0 | 0 |
| C12:0 | 0.2 |
| C15:0 | 0 |
| C14:0 | 0.1 |
| C16:0 | 6 |
| C16:1C | 0.1 |
| C16:1T | 0 |
| C17:0 | 0.1 |
| C18:0 | 32.1 |
| C18:1 | 51.9 |
| C18:1T | 0.1 |
| C18:1C | 51.9 |
| C18:2 | 8 |
| C18:2T | 0 |
| C18:2C | 8 |
| C18:3 | 0.3 |
| C18:3T | 0.1 |
| C18:3C | 0.2 |
| Total Trans | 0.2 |
| C20:0 | 0.7 |
| C20:1C | 0.3 |
| C20:20 | 0 |
| C22:0 | 0.1 |
| C22:1 | 0 |
| C22:1T | 0 |
| C22:1C | 0 |
| C24:0 | 0 |
| C24:1C | 0 |

In the above table:
Cx:y refers to a fatty acid having x carbon atoms and y double bonds; and
C refers to cis fatty acids and T to trans fatty acids; levels determined by GC-FAME (ISO 12966-2 and ISO 12966-4).

358.5 g free fatty acids after distillation was esterified with 89.6 g glycerol in the presence of immobilized lipase originating from Candida antarctica B (Novozym® 435). When the reaction was completed, after approximatively 24 hours, the product was filtered. Then this product was bleached and deodorised at low temperature (mild refining) to obtain the emulsifier composition, which has the following composition, w/w (Table 2):

| | |
|---|---|
| C8:0 | 0 |
| C10:0 | 0 |
| C12:0 | 0.2 |
| C15:0 | 0 |
| C14:0 | 0.1 |
| C16:0 | 6.1 |
| C16:1C | 0.1 |
| C16:1T | 0 |
| C17:0 | 0.1 |
| C18:0 | 32.7 |
| C18:1 | 52 |
| C18:1T | 0.1 |
| C18:1C | 51.9 |
| C18:2 | 7.1 |
| C18:2T | 0 |
| C18:2C | 7.1 |
| C18:3 | 0.2 |
| C18:3T | 0.1 |
| C18:3C | 0.2 |
| Total Trans | 0.1 |
| C20:0 | 0.8 |
| C20:1C | 0.3 |
| C20:2C | 0 |
| C22:0 | 0 |
| C22:1 | 0 |
| C22:1T | 0 |
| C22:1C | 0 |
| C24:0 | 0.1 |
| C24:1C | 0 |
| Triglyceride | 22.6 |
| Diglyceride | 47.7 |
| Monoglyceride | 26.5 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride were determined by HPLC (ISO 18395: 2005(E)).

### Example 2

The distilled fatty acid product obtained in Example 1 was dry-fractionated using a lab-scale crystallizer. The fractionation was performed at 30° C. to 45° C. The free fatty acids were first heated to 70° C. and then cooled down to 30° C. to 45° C. in 5 to 10 hours and held at this temperature for 5 to 10 hours. The crystals formed were separated by means of filter pressing. The slurry was pressed using the following program: increase pressure from 0 to 24 bar in 60 minutes and squeeze at 24 bar for 30 minutes. A high oleic acid olein fraction was obtained. The analytical results are shown in Table 3.

| | |
|---|---|
| C8:0 | 0 |
| C10:0 | 0 |
| C12:0 | 0.2 |
| C15:0 | 0 |
| C14:0 | 0.1 |
| C16:0 | 6.2 |
| C16:1C | 0.1 |
| C16:1T | 0 |
| C17:0 | 0.1 |
| C18:0 | 13.2 |
| C18:1 | 68.7 |
| C18:1T | 0.1 |
| C18:1C | 68.6 |
| C18:2 | 9.7 |
| C18:2T | 0 |
| C18:2C | 9.6 |
| C18:3 | 0.3 |
| C18:3T | 0.1 |
| C18:3C | 0.2 |
| Total Trans | 0.2 |
| C20:0 | 0.8 |
| C20:1C | 0.4 |
| C20:2C | 0 |
| C22:0 | 0.1 |
| C22:1 | 0 |
| C22:1T | 0 |
| C22:1C | 0 |
| C24:0 | 0 |
| C24:1C | 0 |

In the above table:
Cx:y refers to a fatty acid having x carbon atoms and y double bonds; and
C refers to cis fatty acids and T to trans fatty acids; levels determined by GC-FAME (ISO 12966-2 and ISO 12966-4).

205.5 g of the high oleic acid olein fraction was esterified with 94.5 g glycerol in the presence of zinc chloride at 190° C. to 200° C. and 50 to 150 mbar. When the reaction was completed, after approximatively 1 hour, the product was filtered and then mildly refined. After mild refining, an emulsifier composition was obtained, which had the following composition, w/w (Table 4):

| | |
|---|---|
| C8:0 | 0 |
| C10:0 | 0 |
| C12:0 | 0.1 |
| C15:0 | 0 |
| C14:0 | 0.1 |
| C16:0 | 6.1 |
| C16:1C | 0.1 |
| C16:1T | 0 |
| C17:0 | 0.1 |
| C18:0 | 13.2 |
| C18:1 | 68.9 |
| C18:1T | 0.1 |
| C18:1C | 68.8 |
| C18:2 | 9.5 |
| C18:2T | 0 |
| C18:2C | 9.4 |
| C18:3 | 0.3 |
| C18:3T | 0.1 |
| C18:3C | 0.2 |
| Total Trans | 0.2 |
| C20:0 | 0.8 |
| C20:1C | 0.4 |
| C20:2C | 0.1 |
| C22:0 | 0.1 |
| C22:1 | 0 |
| C22:1T | 0 |
| C22:1C | 0 |
| C24:0 | 0.1 |
| C24:1C | 0 |
| Triglyceride | 11.1 |
| Diglyceride | 46.5 |
| Monoglyceride | 38.1 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride were determined by HPLC (ISO 18395: 2005(E)).

### Example 3

600 g of the high oleic acid olein fraction obtained such as in Example 2 was esterified with 150 g glycerol in the presence of immobilized lipase originating from Candida antarctica B (Novozym® 435). When the reaction was completed, after approximatively 24 hours, the product was filtered. Then this product was bleached and deodorised at low temperature (mild refining) to obtain the emulsifier composition, which has the following composition, w/w (Table 5):

| | |
|---|---|
| C8:0 | 0 |
| C10:0 | 0 |
| C12:0 | 0.2 |
| C15:0 | 0 |
| C14:0 | 0.1 |
| C16:0 | 6.4 |
| C16:1C | 0.1 |
| C16:1T | 0 |
| C17:0 | 0.1 |
| C18:0 | 13.1 |
| C18:1 | 68.7 |
| C18:1T | 0.1 |
| C18:1C | 68.6 |
| C18:2 | 9.6 |
| C18:2T | 0 |
| C18:2C | 9.6 |
| C18:3 | 0.3 |
| C18:3T | 0.1 |
| C18:3C | 0.2 |
| Total Trans | 0.2 |
| C20:0 | 0.7 |
| C20:1C | 0.4 |
| C20:2C | 0 |
| C22:0 | 0.1 |
| C22:1 | 0 |
| C22:1T | 0 |
| C22:1C | 0 |
| C24:0 | 0.1 |
| C24:1C | 0 |
| Triglyceride | 22.6 |
| Diglyceride | 48.4 |
| Monoglyceride | 25.2 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride determined by HPLC (ISO 18395: 2005(E)).

The product containing monoglycerides was distilled in order to separate monoglyceride from triglycerides and diglycerides by means of short path distillation at a temperature of about 180° C. and a pressure of about 1×10⁻² mbar. The concentrated monoglyceride fraction was collected as distillate. The concentrated monoglyceride emulsifier has the following composition, w/w (Table 6):

| | |
|---|---|
| C8:0 | 0 |
| C10:0 | 0 |
| C12:0 | 0.2 |
| C15:0 | 0 |
| C14:0 | 0.2 |
| C16:0 | 6.9 |
| C16:1C | 0.1 |
| C16:1T | 0 |
| C17:0 | 0.1 |
| C18:0 | 13.8 |
| C18:1 | 67.7 |
| C18:1T | 0.1 |
| C18:1C | 67.7 |
| C18:2 | 9.4 |
| C18:2T | 0 |
| C18:2C | 9.4 |
| C18:3 | 0.3 |
| C18:3T | 0.1 |
| C18:3C | 0.2 |
| Total Trans | 0.2 |
| C20:0 | 0.6 |
| C20:1C | 0.3 |
| C20:2C | 0 |
| C22:0 | 0 |
| C22:1 | 0 |
| C22:1T | 0 |
| C22:1C | 0 |
| C24:0 | 0 |
| C24:1C | 0 |
| Triglyceride | 0.6 |
| Diglyceride | 2.6 |
| Monoglyceride | 92.1 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride were determined by HPLC (ISO 18395: 2005(E)).

### Example 4

The distilled fatty acid product obtained such as in Example 1 was dry-fractionated using a lab-scale crystallizer. The fractionation was performed at 30° C. to 45° C. The free fatty acids were first heated to 70° C. and then cooled down to 30° C. to 45° C. in 5 to 10 hours and held at this temperature for 5 to 10 hours. The crystals formed were separated by means of filter pressing. The slurry was pressed using the following program: increase pressure from 0 to 24 bar in 60 minutes and squeeze at 24 bar for 30 minutes. A high stearic acid stearin fraction was obtained. The analytical results are shown in Table 7.

| | |
|---|---|
| C8:0 | 0 |
| C10:0 | 0 |
| C12:0 | 0.2 |
| C15:0 | 0 |
| C14:0 | 0.1 |
| C16:0 | 4.8 |
| C16:1C | 0 |
| C16:1T | 0 |
| C17:0 | 0.1 |
| C18:0 | 70.8 |
| C18:1 | 18.9 |
| C18:1T | 0 |
| C18:1C | 18.9 |
| C18:2 | 3.2 |
| C18:2T | 0 |
| C18:2C | 3.2 |
| C18:3 | 0.1 |
| C18:3T | 0 |
| C18:3C | 0.1 |
| Total Trans | 0 |
| C20:0 | 1.5 |
| C20:1C | 0.1 |
| C20:2C | 0 |
| C22:0 | 0.1 |
| C22:1 | 0 |
| C22:1T | 0 |
| C22:1C | 0 |
| C24:0 | 0 |
| C24:1C | 0 |

In the above table:
Cx:y refers to a fatty acid having x carbon atoms and y double bonds; and
C refers to cis fatty acids and T to trans fatty acids; levels determined by GC-FAME (ISO 12966-2 and ISO 12966-4).

301.6 g high stearic acid stearin fraction was esterified with 75.4 g glycerol in the presence of immobilized lipase originating from Candida antarctica B (Novozym® 435). When the reaction was completed, after approximatively 24 hours, the product was filtered. Then this product was bleached and deodorised at low temperature (mild refining) to obtain the emulsifier composition, which has the following composition, w/w (Table 8):

| | |
|---|---|
| C8:0 | 0 |
| C10:0 | 0 |
| C12:0 | 0.2 |
| C15:0 | 0 |
| C14:0 | 0.1 |
| C16:0 | 4.6 |
| C16:1C | 0 |
| C16:1T | 0 |
| C17:0 | 0.1 |
| C18:0 | 71.1 |
| C18:1 | 18.9 |
| C18:1T | 0 |
| C18:10 | 18.8 |
| C18:2 | 3.1 |
| C18:2T | 0 |
| C18:2C | 3.1 |
| C18:3 | 0.1 |
| C18:3T | 0 |
| C18:3C | 0.1 |
| Total Trans | 0.1 |
| C20:0 | 1.5 |
| C20:1C | 0.1 |
| C20:2C | 0 |
| C22:0 | 0.1 |
| C22:1 | 0 |
| C22:1T | 0 |
| C22:1C | 0 |
| C24:0 | 0 |
| C24:1C | 0 |
| Triglyceride | 21 |
| Diglyceride | 51.6 |
| Monoglyceride | 26.1 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride determined by HPLC (ISO 18395: 2005(E)).

### Comparative Example

271.7 g refined shea butter was reacted with 26.9 g glycerol in the presence of 1.5 g potassium hydroxide flakes at 200° C. and under vacuum lower than 100mbar. When the reaction was completed, after approximatively 30 minutes, the product was bleached and deodorized in order to obtain a comparative example, which has the following composition

(Table 9):

| | |
|---|---|
| C8:0 | 0 |
| C10:0 | 0 |
| C12:0 | 0.4 |
| C15:0 | 0 |
| C14:0 | 0.2 |
| C16:0 | 4 |
| C16:1C | 0.1 |
| C16:1T | 0 |
| C17:0 | 0.1 |
| C18:0 | 43.9 |
| C18:1 | 43.3 |
| C18:1T | 0.1 |
| C18:1C | 43.2 |
| C18:2 | 5.8 |
| C18:2T | 0.1 |
| C18:2C | 5.8 |
| C18:3 | 0.2 |
| C18:3T | 0.1 |
| C18:3C | 0.1 |
| Total Trans | 0.2 |
| C20:0 | 1.4 |
| C20:1C | 0.2 |
| C20:2C | 0 |
| C22:0 | 0.1 |
| C22:1 | 0 |
| C22:1T | 0 |
| C22:1C | 0 |
| C24:0 | 0.1 |
| C24:1C | 0 |
| Triglyceride | 18.3 |
| Diglyceride | 42.6 |
| Monoglyceride | 25.7 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride determined by HPLC (ISO 18395: 2005(E)).

### Example 5

An emulsion stability test was performed in order to evaluate the functionality of the emulsifiers. Besides the Comparative ExaMple, a commercial emulsifier product Durem 35NG from 101 Loders Croklaan US was also included, which has the following composition (Table 10):

| | |
|---|---|
| C8:0 | 0 |
| C10:0 | 0 |
| C12:0 | 0.3 |
| C15:0 | 0.1 |
| C14:0 | 1.1 |
| C16:0 | 42.3 |
| C16:1C | 0.2 |
| C16:1T | 0 |
| C17:0 | 0.1 |
| C18:0 | 4.6 |
| C18:1 | 41.1 |
| C18:1T | 0.1 |
| C18:1C | 41 |
| C18:2 | 9.2 |
| C18:2T | 0.6 |
| C18:2C | 8.6 |
| C18:3 | 0.2 |
| C18:3T | 0.1 |
| C18:3C | 0.1 |
| Total Trans | 0.8 |
| C20:0 | 0.4 |
| C20:1C | 0.2 |
| C20:2C | 0 |
| C22:0 | 0.1 |
| C22:1 | 0 |
| C22:1T | 0 |
| C22:1C | 0 |
| C24:0 | 0.1 |
| C24:1C | 0 |
| Triglyceride | 10.7 |
| Diglyceride | 48.3 |
| Monoglyceride | 40.2 |

In the above table:
Cx:y refers to a fatty acid having x carbon atoms and y double bonds;
C refers to cis fatty acids and T to trans fatty acids; levels determined by GC-FAME (ISO 12966-2 and ISO 12966-4); and
Triglyceride/Diglyceride/Monoglyceride determined by HPLC (ISO 18395: 2005(E)).

0.8 g of the emulsifier of each of Example 1, Example 2, Example 4, the Comparative Example and Durem 35NG respectively were totally dissolved in 80 g rapeseed oil. Each mixture was put into a 120 ml glass bottle and mixed with a propeller with four symmetrical square blades of 0.8 cm each at a speed of 750 rpm. When the temperature of each mixture is at approximatively 30° C., 20ml demineralized water was gently added into each mixture within 15 seconds. Each emulsion was further mixed at room temperature under the same mixing conditions for 1 minute. Then, each emulsion was poured into a 100 ml glass graduated cylinder at room temperature. After 30 minutes, the volume of water layer was read respectively in order to evaluate the emulsion stability. One control test was done without any emulsifier. The stability was calculated with the following formula:

$$Emulsion stability \% = \frac{20 - Volume of water layer after separation }{20} \%$$

The results are shown in the following table (Table 11):

| | | | | | Compar- | |
|---|---|---|---|---|---|---|
| | Con- | Exam- | Exam- | Exam- | ative | Durem |
| | trol | ple 1 | ple 2 | ple 4 | Example | 35NG |
| Volume of | 17 | 9 | 6 | 10 | 15 | 13 |
| water layer | | | | | | |
| after sepa- | | | | | | |
| ration (ml) | | | | | | |
| Emulsion | 15% | 55% | 70% | 50% | 25% | 35% |
| stability % | | | | | | |

Example 1, Example 2 and Example 4 show better emulsion stability than the Comparative Example and Durem 35 NG.

### Example 6

An emulsion stability test was performed in order to evaluate the functionality of the emulsifiers. A commercial emulsifier product Dimodan HP MB from Danisco, DuPont Group was included, which has the following composition (Table 12):

| | |
|---|---|
| C8:0 | 0 |
| C10:0 | 0 |
| C12:0 | 0.3 |
| C15:0 | 0.1 |
| C14:0 | 1.2 |
| C16:0 | 55.2 |
| C16:1C | 0 |
| C16:1T | 0 |
| C17:0 | 0.1 |
| C18:0 | 42.4 |
| C18:1 | 0.1 |
| C18:1T | 0 |
| C18:1C | 0 |
| C18:2 | 0 |
| C18:2T | 0 |
| C18:2C | 0 |
| C18:3 | 0 |
| C18:3T | 0 |
| C18:3C | 0 |
| Total Trans | 0 |
| C20:0 | 0.5 |
| C20:1C | 0 |
| C20:2C | 0 |
| C22:0 | 0.1 |
| C22:1 | 0 |
| C22:1T | 0 |
| C22:1C | 0 |
| C24:0 | 0.1 |
| C24:1C | 0 |
| Triglyceride | 0.1 |
| Diglyceride | 2.5 |
| Monoglyceride | 97.1 |

0.4 g emulsifier of each of Example 3 and Dimodan HP MB respectively was totally dissolved in 80 g rapeseed oil. Each mixture was put into a 120 ml glass bottle and mixed with a propeller with four symmetrical square blades of 0.8 cm each at a speed of 750 rpm. When the temperature of each mixture is at approximatively 30° C., 20ml demineralized water was gently added into each mixture within 15 seconds. Each emulsion was further mixed at room temperature under the same mixing conditions for 1 minute. Then, each emulsion was poured into a 100 ml glass graduated cylinder at room temperature. After 30 minutes, the volume of water layer was read respectively in order to evaluate the emulsion stability. One control test was done without any emulsifier. The stability was calculated with the following formula:

$$Emulsion stability \% = \frac{20 - Volume of water layer after separation }{20} \%$$

The results are shown in the following table (Table 13):

| | Control | Example 3 | Dimodan HP MB |
|---|---|---|---|
| Volume of water layer | 17 | 13 | 15 |
| after separation (ml) | | | |
| Emulsion stability % | 15% | 35% | 25% |

Example 3 shows better emulsion stability than Dimodan HP MB.

1. A method for preparing a non-hydrogenated, non-palm emulsifier composition, comprising the steps of:
providing a fatty acid composition comprising at least 80% by weight free fatty acids, and
reacting the fatty acid composition with glycerol,
wherein the emulsifier composition comprises:
at least 20% by weight monoglycerides;
less than 60% by weight of diglycerides; and
and from 0-80% by weight triglycerides,
wherein the weight % is with respect to the total of the monoglycerides, diglycerides and triglycerides, and
wherein fatty acid residues bound to the monoglycerides, diglycerides and triglycerides in the emulsifier composition comprise:
from 5% to 80% by weight stearic acid (C18:0);
from 10% to 80% by weight oleic acid (C18:1); and
from 1% to 10% by weight palmitic acid (C16:0),
based on the total weight of C8 to C24 fatty acids.

2. The method according to claim 1, wherein the fatty acid composition is obtained from shea butter, shea olein, shea stearin, sal butter, sal stearin, sal olein, or mixtures thereof.

3. The method according to claim 1, wherein the reaction of the fatty acid composition with glycerol is carried out in the presence of an enzymatic catalyst.

4. The method according to claim 1, wherein the reaction of the fatty acid composition with glycerol is carried out in the presence of an inorganic salt.

5. A non-hydrogenated, non-palm emulsifier composition comprising:
at least 20% by weight monoglycerides;
less than 60% by weight of diglycerides; and
from 0-80% by weight triglycerides,
wherein the weight % is with respect to the total of the monoglycerides, diglycerides and triglycerides, and
wherein fatty acid residues bound to the monoglycerides, diglycerides and triglycerides in the emulsifier composition comprise:
from 5% to 40% by weight stearic acid (C18:0);
from 45% to 80% by weight oleic acid (C18:1); and
from 1% to 10% by weight palmitic acid (C16:0),
based on the total weight of C8 to C24 fatty acids.

6. The composition according to claim 5, wherein the weight ratio of stearic acid to oleic acid is from 1:8 to 9:10.

7. The composition according to claim 5, comprising from 20% to 50% by weight monoglycerides.

8. The composition according to claim 5, comprising from 30% to 60% by weight diglycerides.

9. The composition according to claim 5 comprising: at least 85% by weight monoglycerides; less than 10% by weight of diglycerides; and less than 5% by weight triglycerides.

10. The composition according to claim 5, comprising from 1% to 12% by weight linoleic acid (C18:2).

11. The composition according to claim 5, comprising from 10% to 35% by weight stearic acid.

12. The composition according to claim 5, comprising from 50% to 70% by weight oleic acid.

13. The composition according to claim 5, comprising from 4.5% to 8% by weight palmitic acid.

14. A non-hydrogenated, non-palm emulsifier composition comprising:
at least 20% by weight monoglycerides;
less than 60% by weight of diglycerides; and
and from 0-80% by weight triglycerides,
wherein the weight % is with respect to the total of the monoglycerides, diglycerides and triglycerides, and
wherein fatty acid residues bound to the monoglycerides, diglycerides and triglycerides in the emulsifier composition comprise:
from 50% to 90% by weight stearic acid (C18:0);
from 10% to 30% by weight oleic acid (C18:1); and
from 1% to 10% by weight palmitic acid (C16:0),
based on the total weight of C8 to C24 fatty acids.

15. A bakery or confectionery product comprising the composition according to claim 5.

16. The product according to claim 15, wherein the product is selected from the group consisting of puff pastry, cake, Danish rolls or water-based fillings.

17. The method according to claim 2, wherein the fatty acid composition is obtained from shea olein.

18. The method according to claim 3, wherein the enzymatic catalyst is a lipase.

19. The method according to claim 4, wherein the inorganic salt is zinc chloride.

20. The composition according to claim 5, comprising from 22% to 40% by weight monoglycerides.
